(19) United States
(12) Patent Application Publication
(10) Pub. No.: US 2024/0425883 A1
(43) Pub. Date: Dec. 26, 2024

(54) RESPIRATORY DELIVERY OF THERAPEUTIC AGENTS

(71) Applicant: Aldevron, LLC, South Fargo, ND (US)

(72) Inventors: Rachelle PRANTIL-BAUN, South Fargo, ND (US); Robert FARRA, Cambridge, MA (US); Jake CEMAZAR, Cambridge, MA (US); Richard BOUCHER, Chapel Hill, NC (US); Lihua HE, Chapel Hill, NC (US); Takafumi KATO, Chapel Hill, NC (US); Scott RANDELL, Chapel Hill, NC (US)

(21) Appl. No.: 18/700,975

(22) PCT Filed: Oct. 14, 2022
(86) PCT No.: PCT/US2022/078165

§ 371 Date: Apr. 12, 2024

Related U.S. Application Data
(60) Provisional application No. 63/256,416, filed on Oct. 15, 2021.
(60) Provisional application No. 63/388,794, filed on Jul. 13, 2022.

(51) Int. Cl. C12N 15/87; A61K 48/00; A61N 1/32; C07K 14/705; C12N 15/85

(52) U.S. Cl. CPC C12N 15/87; A61K 48/0016; A61K 48/005; A61N 1/327; C07K 14/705; C12N 15/85

(57) ABSTRACT

Provided herein are methods of delivering therapeutic agents, such as synthetic circular DNA vectors, to target airway cells (e.g., airway epithelial cells). Such methods can be useful in treating respiratory diseases or disorders, such as cystic fibrosis.

### CROSS-REFERENCE

This application claims the benefit of U.S. Provisional Application Nos. 63/256,416, filed on Oct. 15, 2021 and 63/388,794 filed on Jul. 13, 2022, which are incorporated herein by reference in their entirety.

### FIELD OF THE INVENTION

In general, the invention features methods of delivering therapeutic agents to airway cells.

### BACKGROUND

Gene therapy is emerging as a promising approach to treat a wide variety of diseases and disorders in human patients. However, progress has been restricted in certain tissues due to inaccessibility and protective physiology. For instance, gene delivery to airway cells (e.g., lung cells) has proven challenging, in part due to barriers at the luminal surface. Thus, there is a need in the field for improved methods of delivering therapeutic agents (e.g., gene delivery) to target airway cells.

### SUMMARY OF THE INVENTION

Provided herein are methods of delivering therapeutic agents, such as synthetic circular DNA vectors, to target airway cells (e.g., airway epithelial cells). Such methods can be useful in treating respiratory diseases or disorders, such as cystic fibrosis.

In one aspect, provided is a method of expressing a sequence of interest (e.g., a therapeutic sequence) in a target airway cell. The method includes the following steps: (i) exposing the target airway cell to a synthetic circular DNA vector comprising the sequence of interest (e.g., a therapeutic sequence), wherein the target airway cell is in electrical communication with an electrode; and (ii) transmitting one or more pulses of electrical energy through the electrode at conditions suitable for electrotransfer of the synthetic circular DNA vector into the target airway cell, thereby expressing the sequence of interest (e.g., a therapeutic sequence). In some embodiments, the synthetic circular DNA vector comprises a sequence of interest (e.g., a therapeutic sequence) having a 5′ end and a 3′ end, wherein the 5′ end is connected to (e.g., directly connected to, without any intervening sequences) the 3′ end by a non-bacterial sequence. In some embodiments, the non-bacterial sequence corresponds to a restriction enzyme cut site overhang. In some embodiments, the non-bacterial sequence consists of a sequence corresponding to a restriction enzyme overhang. In some embodiments, the synthetic circular DNA vector is a naked synthetic circular DNA vector. In some embodiments, the synthetic circular DNA vector is encapsulated in, or associated with, a particulate structure (e.g., a liposome). In some embodiments, the sequence of interest (e.g., a therapeutic sequence) comprises a therapeutic protein-encoding sequence, such as cystic fibrosis transmembrane receptor (CFTR). In some embodiments, the sequence of interest (e.g., a therapeutic sequence) comprises one or more regulatory elements. In some embodiments, the sequence of interest (e.g., a therapeutic sequence) comprises a therapeutic nucleic acid.

In some embodiments of any of the preceding methods, the target airway cell is an airway epithelial cell. In some embodiments, the target airway cell is a lung cell. In some embodiments, the target airway cell is a nasal epithelial cell, a tracheal cell, bronchial cell, bronchiolar cell, or an alveolar cell.

In some embodiments of any of the preceding methods, the conditions suitable for electrotransfer of the therapeutic agent into the target airway cell comprise a voltage at the target lung cell of less than 2,400 V. In some embodiments, the voltage at the target airway cell is between 10 V and 2,400 V (e.g., between 100 V and 2,400 V, e.g., from 100 V to 500 V, from 500 V to 1,000 V, from 1,000 V to 1,500 V, or from 1,500 V to 2,000 V). In some embodiments, the voltage at the target airway cell is between 1600 V and 2,400 V, between 1800 V and 2,400 V, or between 2,000 V and 2,400 V). In some embodiments, 1-10 (e.g., 1-6) pulses of electrical energy are transmitted. In some embodiments, the total number of pulses of electrical energy are transmitted within 1-20 seconds. In some embodiments, a single pulse (i.e., one and only one pulse) of electrical energy is transmitted. In other embodiments, exactly two pulses of electrical energy is transmitted. In some embodiments, the one or more pulses of electrical energy are square waveforms. In some embodiments, the one or more pulses of electrical energy have an amplitude from 10 V to 10,000V, e.g., from 100 V to 5,000 V, e.g., from 100 V to 500 V, from 500 V to 1,000 V, from 1,000 V to 1,500 V, or from 1,500 V to 2,000 V. In some embodiments, each of the pulses of electrical energy is from 0.01 to 200 milliseconds in duration. In some embodiments, each of the pulses of electrical energy is from 1 to 100 milliseconds in duration, e.g., 10 to 50 milliseconds in duration (e.g., about 20 milliseconds in duration). In some embodiments, the total duration of all of the pulses of electrical energy is from 1 to 100 milliseconds in duration, e.g., 10 to 50 milliseconds (e.g., about 20 milliseconds in total duration). In some embodiments, the electrode is a monopolar electrode. In other embodiments, the electrode is part of a bipolar electrode configuration. In some embodiments, the electrode is within 10 cm of the target airway cell. For example, in some embodiments, the electrode is within 1 cm of the target airway cell (e.g., within 5 mm, within 4 mm, or within 3 mm).

In some embodiments, the expression level of the sequence of interest (e.g., a therapeutic sequence) from the synthetic circular DNA vector is greater than expression level of a sequence of interest (e.g., a therapeutic sequence) from a reference plasmid DNA vector under analogous conditions. For instance, in some embodiments, the expression level of the sequence of interest (e.g., a therapeutic sequence) from the synthetic circular DNA vector is at least 10% greater than expression level of a sequence of interest (e.g., a therapeutic sequence) from a reference plasmid DNA vector under analogous conditions. Additionally, or alternatively, the expression persistence of the sequence of interest (e.g., a therapeutic sequence) from the synthetic circular DNA vector may be greater than expression persistence of a sequence of interest (e.g., a therapeutic sequence) from a reference plasmid DNA vector under analogous conditions.

In some embodiments, the target airway cell has a mutation in a gene (e.g., CFTR) correctable by the therapeutic sequence.

In some embodiments, step (ii) is performed in situ in an individual by inserting a catheter comprising the electrode into an airway lumen of the individual. In some embodiments, the airway lumen is trachea, bronchi, or bronchiole. In some embodiments, the individual has been diagnosed with a disease or disorder. In some embodiments, the disease or disorder is a monogenic respiratory disease, e.g., cystic fibrosis.

In some embodiments, the synthetic circular DNA vector has been administered to the individual locally. In some embodiments, the local administration was intranasal administration or intramuscular administration. In some embodiments, the local administration was by flood, spray, or aerosolization of the synthetic circular DNA vector. In some embodiments, the synthetic circular DNA vector has been administered to the individual systemically. In some embodiments, the systemic administration was intravenous administration.

In some embodiments, the method further comprises administering the synthetic circular DNA vector. In some embodiments, the synthetic circular DNA vector is administered to the individual locally. In some embodiments, the local administration is intranasal administration or intramuscular administration. In some embodiments, the local administration is by flood, spray, or aerosolization of the synthetic circular DNA vector. In some embodiments, the synthetic circular DNA vector is administered to the individual systemically. In some embodiments, the systemic administration is intravenous administration. In some embodiments, partial airway epithelial ablation therapy has preceded the administration of the synthetic circular DNA vector. In some embodiments, the partial airway epithelial ablation was administered using a pulsed electric field. In some embodiments, the method further comprises administering the partial airway epithelial ablation therapy.

In some embodiments of any of the preceding methods, the target airway cell is a human cell.

In another aspect, the invention provides a method of delivering a therapeutic agent (e.g., a therapeutic sequence encoding a protein (e.g., as part of a synthetic circular DNA vector) or a therapeutic nucleic acid (e.g., a CRISPR molecule)) to a target airway cell in an individual, the method comprising: (i) inserting a catheter comprising a monopolar electrode into an airway lumen of the individual; (ii) positioning the electrode within a target region of the airway lumen comprising the target airway cell and the therapeutic agent; (iii) while the electrode is within the target region, transmitting one or more pulses of electrical energy through the electrode at conditions suitable for electrotransfer of the therapeutic agent into the target airway cell. In some embodiments, the individual has been treated with a partial airway epithelial ablation therapy.

In another aspect, the invention provides a method of delivering a therapeutic agent (e.g., a therapeutic sequence encoding a protein (e.g., as part of a synthetic circular DNA vector) or a therapeutic nucleic acid (e.g., a CRISPR molecule)) to a target airway cell in an individual that has been treated with a partial airway epithelial ablation therapy, the method comprising: (i) inserting a catheter comprising an electrode into an airway lumen of the individual; (ii) positioning the electrode within a target region of the airway lumen comprising the target airway cell and the therapeutic agent; and (iii) while the electrode is within the target region, transmitting one or more pulses of electrical energy through the electrode at conditions suitable for electrotransfer of the therapeutic agent into the target airway cell.

In some embodiments, the therapeutic agent comprises a nucleic acid vector comprising a sequence of interest (e.g., a therapeutic sequence). In some embodiments, the nucleic acid vector is a non-viral nucleic acid vector. In some embodiments, the nucleic acid vector is a naked nucleic acid vector. In some embodiments, the nucleic acid vector is encapsulated in, or associated with, a particulate structure, such as a liposome. In some embodiments, the nucleic acid vector is a DNA vector. In some embodiments, the DNA vector is a synthetic DNA vector. In some embodiments, the synthetic DNA vector is a synthetic circular DNA vector. In some embodiments, the sequence of interest (e.g., a therapeutic sequence) has a 5′ end and a 3′ end, wherein the 5′ end is connected to the 3′ end by a non-bacterial sequence corresponding to a restriction enzyme cut site overhang. In some embodiments, the non-bacterial sequence consists of a sequence corresponding to a restriction enzyme overhang. In some embodiments, the expression level of the sequence of interest (e.g., a therapeutic sequence) from the synthetic circular DNA vector is greater than expression level of a sequence of interest (e.g., a therapeutic sequence) from a reference plasmid DNA vector under analogous conditions. In some embodiments, the expression level of the sequence of interest (e.g., a therapeutic sequence) from the synthetic circular DNA vector is at least 10% greater than expression level of a sequence of interest (e.g., a therapeutic sequence) from a reference plasmid DNA vector under analogous conditions. In some embodiments, the expression persistence of the sequence of interest (e.g., a therapeutic sequence) from the synthetic circular DNA vector is greater than expression persistence of a sequence of interest (e.g., a therapeutic sequence) from a reference plasmid DNA vector under analogous conditions.

In some embodiments, the sequence of interest (e.g., a therapeutic sequence) comprises a therapeutic protein-encoding sequence, such as CFTR. In some embodiments, the sequence of interest (e.g., a therapeutic sequence) comprises one or more regulatory elements. In some embodiments, the sequence of interest (e.g., a therapeutic sequence) comprises a therapeutic nucleic acid (such as a CRISPR molecule). In some embodiments, the target airway cell is an airway epithelial cell. In some embodiments, the target airway cell is a lung cell. In some embodiments, the target airway cell is a nasal epithelial cell, a tracheal cell, bronchial cell, bronchiolar cell, or an alveolar cell.

In some embodiments, the conditions suitable for electrotransfer of the therapeutic agent into the target airway cell comprise a voltage at the target lung cell of less than 2,400 V. For instance, the voltage at the target airway cell may be between 10 V and 2,400 V (e.g., between 100 V and 2,400 V, e.g., from 100 V to 500 V, from 500 V to 1,000 V, from 1,000 V to 1,500 V, or from 1,500 V to 2,000 V). In some embodiments, the voltage at the target airway cell is between 1600 V and 2,400 V, between 1800 V and 2,400 V, or between 2,000 V and 2,400 V). In some embodiments, 1-10 (e.g., 1-6) pulses of electrical energy are transmitted. In some embodiments, the total number of pulses of electrical energy are transmitted within 1-20 seconds. In some embodiments, a single pulse of electrical energy is transmitted. In some embodiments, exactly two pulses of electrical energy are transmitted. In some embodiments, the one or more pulses of electrical energy are square waveforms. In some embodiments, the one or more pulses of electrical energy have an amplitude from 10 V to 10,000 V, e.g., from 100 V to 5,000 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V). In some embodiments, each of the pulses of electrical energy is from 0.01 to 200 milliseconds in duration. In some embodiments, each of the pulses of electrical energy is from 1 to 100 milliseconds in duration, e.g., 10 to 50 milliseconds in duration. In some embodiments, the total duration of all of the pulses of electrical energy is from 1 to 100 milliseconds, e.g., 10 to 50 milliseconds.

In some embodiments, the electrode is a monopolar electrode. In other embodiments, the electrode is part of a bipolar electrode configuration. In some embodiments, the electrode is within 10 cm of the target airway cell. In some embodiments, the electrode is within 1 cm of the target airway cell (e.g., within 5 mm, within 4 mm, or within 3 mm).

In some embodiments, the target airway cell has a mutation in a gene correctable by the therapeutic sequence. In some embodiments, the airway lumen is trachea, bronchi, or bronchiole. In some embodiments, the individual has been diagnosed with a disease or disorder. In some embodiments, the disease or disorder is a monogenic respiratory disease. In some embodiments, the monogenic respiratory disease is cystic fibrosis.

In some embodiments, the synthetic circular DNA vector has been administered to the individual locally. In some embodiments, the local administration was intranasal administration or intramuscular administration. In some embodiments, the local administration was by flood, spray, or aerosolization of the synthetic circular DNA vector. In some embodiments, the synthetic circular DNA vector has been administered to the individual systemically. In some embodiments, the systemic administration was intravenous administration.

In some embodiments, the method further comprises administering the synthetic circular DNA vector. In some embodiments, the synthetic circular DNA vector is administered to the individual locally. In some embodiments, the local administration is intranasal administration or intramuscular administration. In some embodiments, the local administration is by flood, spray, or aerosolization of the synthetic circular DNA vector. In some embodiments, the synthetic circular DNA vector is administered to the individual systemically. In some embodiments, the systemic administration is intravenous administration.

In some embodiments, the partial airway epithelial ablation therapy was performed using a pulsed electric field.

In some embodiments, the individual is a human.

In another aspect, the invention provides a method of treating a disease or disorder in an individual, the method comprising: (i) positioning an electrode in electrical communication with target airway cells of the individual, wherein the target airway cells are exposed to a therapeutically effective amount of a synthetic circular DNA vector comprising a therapeutic sequence; and (ii) transmitting one or more pulses of electrical energy through the electrode at conditions suitable for electrotransfer of the synthetic circular DNA vector into the target airway cells, thereby expressing the therapeutic sequence and treating the disease or disorder.

In some embodiments, the synthetic circular DNA vector comprises a therapeutic sequence having a 5′ end and a 3′ end, wherein the 5′ end is connected to the 3′ end by a non-bacterial sequence. In some embodiments, the non-bacterial sequence corresponds to a restriction enzyme cut site overhang. In some embodiments, the non-bacterial sequence consists of a sequence corresponding to a restriction enzyme overhang. In some embodiments, the synthetic circular DNA vector is a naked synthetic circular DNA vector. In some embodiments, the synthetic circular DNA vector is encapsulated in, or associated with, a particulate structure, such as a liposome. In some embodiments, the therapeutic sequence comprises a therapeutic protein-encoding sequence. In some embodiments, the therapeutic protein is CFTR. In some embodiments, the therapeutic sequence comprises one or more regulatory elements. In some embodiments, the therapeutic sequence comprises a therapeutic nucleic acid.

In some embodiments, the target airway cell is an airway epithelial cell (e.g., a proximal airway epithelial cell (e.g., a proximal airway epithelial stem cell or a proximal airway epithelial progenitor cell)) or a distal airway epithelial cell). In some embodiments, the target airway cell is a lung cell. In some embodiments, the target airway cell is a nasal epithelial cell, a tracheal cell, bronchial cell, bronchiolar cell, or an alveolar cell.

In some embodiments, the conditions suitable for electrotransfer of the therapeutic agent into the target airway cell comprise a voltage at the target lung cell of less than 2,400 V. In some embodiments, the voltage at the target airway cell is between 10 V and 2,400 V, e.g., from 100 V and 2,400 V, e.g., from 100 V to 500 V, from 500 V to 1,000 V, from 1,000 V to 1,500 V, or from 1,500 V to 2,000 V. In some embodiments, the voltage at the target airway cell is between 1600 V and 2,400 V, between 1800 V and 2,400 V, or between 2,000 V and 2,400 V). In some embodiments, 1-10 (e.g., 1-6) pulses of electrical energy are transmitted. In some embodiments, the total number of pulses of electrical energy are transmitted within 1-20 seconds. In some embodiments, a single pulse of electrical energy is transmitted. In some embodiments, exactly two pulses of electrical energy are transmitted. In some embodiments, the one or more pulses of electrical energy are square waveforms. In some embodiments, the one or more pulses of electrical energy have an amplitude from 10 V to 10,000 V, e.g., from 100 V to 5,000 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V). In some embodiments, each of the pulses of electrical energy is from 0.01 to 200 milliseconds in duration. In some embodiments, each of the pulses of electrical energy is from 1 to 100 milliseconds in duration, e.g., 10 to 50 milliseconds in duration. In some embodiments, the total duration of all of the pulses of electrical energy is from 1 to 100 milliseconds, e.g., 10 to 50 milliseconds.

In some embodiments, the electrode is a monopolar electrode. In other embodiments, the electrode is part of a bipolar electrode configuration. In some embodiments, the electrode is within 10 cm of the target airway cell. In some embodiments, the electrode is within 1 cm of the target airway cell (e.g., within 5 mm, within 4 mm, or within 3 mm).

In some embodiments, the expression level of the therapeutic sequence from the synthetic circular DNA vector is greater than expression level of a therapeutic sequence from a reference plasmid DNA vector under analogous conditions. In some embodiments, the expression level of the therapeutic sequence from the synthetic circular DNA vector is at least 10% greater than expression level of a therapeutic sequence from a reference plasmid DNA vector under analogous conditions. In some embodiments, the expression persistence of the therapeutic sequence from the synthetic circular DNA vector is greater than expression persistence of a therapeutic sequence from a reference plasmid DNA vector under analogous conditions.

In some embodiments, the target airway cell has a mutation in a gene correctable by the therapeutic sequence.

In some embodiments, step (i) comprises inserting a catheter comprising the electrode into an airway lumen of the individual. In some embodiments, the airway lumen is trachea, bronchi, or bronchiole. In some embodiments, the disease or disorder is a monogenic respiratory disease, such as cystic fibrosis.

In some embodiments, the synthetic circular DNA vector has been administered to the individual locally. In some embodiments, the local administration was intranasal administration or intramuscular administration. In some embodiments, the local administration was by flood, spray, or aerosolization of the synthetic circular DNA vector. In some embodiments, the synthetic circular DNA vector has been administered to the individual systemically. In some embodiments, the systemic administration was intravenous administration.

In some embodiments, the method further comprises administering the synthetic circular DNA vector. In some embodiments, the synthetic circular DNA vector is administered to the individual locally. In some embodiments, the local administration is intranasal administration or intramuscular administration. In some embodiments, the local administration is by flood, spray, or aerosolization of the synthetic circular DNA vector. In some embodiments, the synthetic circular DNA vector is administered to the individual systemically. In some embodiments, the systemic administration is intravenous administration.

In some embodiments, partial airway epithelial ablation therapy was administered prior to administration of the synthetic circular DNA vector. In some embodiments, the partial airway epithelial ablation therapy was administered using a pulsed electric field. In some embodiments, the method further comprises administering the partial airway epithelial ablation therapy.

In another aspect, the invention features a method of treating cystic fibrosis in an individual (e.g., a human), the method comprising: (i) inserting a catheter comprising an electrode into an airway lumen of the individual; (ii) positioning the electrode in electrical communication with target airway cells of the individual, wherein the target airway cells are exposed to a therapeutically effective amount of a synthetic circular DNA vector encoding CFTR; and (iii) transmitting one to six pulses of electrical energy through the electrode at a voltage at the target lung cell between 10 V and 2,400 V, e.g., 100 V and 2,400 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V), wherein the total duration of the pulses of electrical energy is from 1 to 100 milliseconds, e.g., 10 to 50 milliseconds, thereby expressing the CFTR and treating the cystic fibrosis.

In another aspect, the invention features a method of treating cystic fibrosis in an individual (e.g., a human), the method comprising: (i) administering to the individual a therapeutically effective amount of a synthetic circular DNA vector encoding CFTR; (ii) inserting a catheter comprising an electrode into an airway lumen of the individual; (iii) positioning the electrode in electrical communication with target airway cells of the individual, wherein the target airway cells are exposed to the synthetic circular DNA vector encoding CFTR; and (iv) transmitting one to six pulses of electrical energy through the electrode at a voltage at the target lung cell between 10 V and 2,400 V, e.g., between 100 V and 2,400 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V), wherein the total duration of the pulses of electrical energy is from 1 to 100 milliseconds, e.g., 10 to 50 milliseconds, thereby expressing the CFTR and treating the cystic fibrosis.

In another aspect, the invention features a method of treating cystic fibrosis in an individual (e.g., a human), the method comprising: (i) administering partial airway epithelial ablation therapy to the individual; (ii) administering to the individual a therapeutically effective amount of a synthetic circular DNA vector encoding CFTR; (iii) inserting a catheter comprising an electrode into an airway lumen of the individual; (iv) positioning the electrode in electrical communication with target airway cells of the individual, wherein the target airway cells are exposed to the synthetic circular DNA vector encoding CFTR; and (v) transmitting one to six pulses of electrical energy through the electrode at a voltage at the target lung cell between 10 V and 2,400 V, e.g., between 100 V and 2,400 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V), wherein the total duration of the pulses of electrical energy is from 1 to 100 milliseconds, e.g., 10 to 50 milliseconds, thereby expressing the CFTR and treating the cystic fibrosis.

### BRIEF DESCRIPTION OF THE DRAWINGS

FIG. 1 is a graph showing expression levels of GFP encoded by a synthetic circular DNA vector (c3DNA) compared to GFP encoded by plasmid DNA vector (P1003) in suspended primary human bronchial epithelial cells across a range of electrotransfer conditions (applied voltage, duration of each pulse (ms), and number of pulses).

FIG. 2 is a set of flow cytometry plots showing gating strategies and results from experiments in which primary human bronchial epithelial cells were transfected with synthetic circular DNA (C³DNA) vectors (top row) compared to plasmid DNA vectors (bottom row) with no pulsed electric field (“no shock”; left-hand column), one 1600 V pulse (middle column), or two 1600 V pulses (right-hand column). On all plots, propidium iodine (a measure of cell death) is on the x axis, and GFP fluorescence is on the y axis.

FIGS. 3A and 3B are bar graphs quantifying viability (FIG. 3A) and percent GFP (EGFP) positivity (FIG. 3B) for each condition indicated.

FIG. 4 is a schematic drawing of a human air liquid interface (ALI) culture of airway epithelial cells. These cultures involve seeding lung epithelial stem cells onto porous membranes of a transwell and subjecting them to ALI, by exposing the cells to medium from the basal side of the chamber. Air and medium components induce the stem cells to differentiate into goblet, ciliated, club, and neuroendocrine (NEC) cells, which comprise a fully functional lung epithelium of the upper airway.

FIGS. 5A and 5B are sets of fluorescent photomicrographs of ALI cultures of human airway epithelial cells. Nuclei staining is shown in blue, GFP (encoded by synthetic circular DNA vectors) is shown in green, phalloidin staining is shown in red, muc5AC (goblet cell) staining is shown in light blue, and a-tubulin (ciliated cell) staining is shown in white. FIG. 5A shows ALI cultures 72 after transfection with synthetic circular DNA vectors without pulsed electric field transmission, whereas FIG. 5B shows ALI cultures 72 after transfection with synthetic circular DNA vectors with pulsed electric field transmission.

FIG. 6 is a photomicrograph showing the microstructure in a section of rabbit tracheal explant with hematoxylin and eosin staining.

FIGS. 7A and 7B are a set of fluorescent photomicrographs of a rabbit tracheal explant transfected with synthetic circular DNA vectors encoding GFP using electrotransfer. Nuclei staining is shown in blue, GFP (encoded by synthetic circular DNA vectors) is shown in green, phalloidin staining is shown in red, and a-tubulin (ciliated cell) staining is shown in white.

FIGS. 8A-8C are a set of fluorescent photomicrographs of rabbit tracheal explant transfected with synthetic circular DNA vectors encoding GFP without and with pulsed electric field. FIG. 8A is a live cell fluorescence image of an exemplary single cell treated with GFP-encoding synthetic circular DNA vector without pulsed electric field (left-hand panel) and with pulsed electric field (right-hand panel). FIGS. 8B and 8C show X-Y (FIG. 8B) and X-Z (FIG. 8C) confocal images of sections with nuclei staining in blue, GFP (encoded by synthetic circular DNA vectors) is shown in green, and F-actin shown in red.

### DETAILED DESCRIPTION

Provided herein are methods of delivering therapeutic agents, such as synthetic circular DNA vectors, to target airway cells (e.g., airway epithelial cells). Such methods can be useful in treating respiratory diseases or disorders, such as cystic fibrosis (e.g., by delivering vectors encoding therapeutic protein-encoding genes, such as CFTR).

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, “exposing” a target cell to a vector refers to contacting the vector to the target cell (e.g., in direct physical contact with the target cell membrane) or to an extracellular region surrounding the target cell (e.g., culture media containing the target cell, or extracellular space in the tissue containing the target cell). A target cell is exposed to a vector if the vector can be electrotransferred to the target cell.

As used herein, “electrotransfer” refers to movement of a molecule (e.g., a nucleic acid, e.g., a naked nucleic acid) across a membrane of a target cell (e.g., from outside to inside the target cell, e.g., a retinal cell) that is caused by transmission of an electric field (e.g., a pulsed electric field) to the microenvironment in which the cell resides. Electrotransfer may occur as a result of electrophoresis, i.e., movement of a molecule (e.g., a nucleic acid, e.g., a naked nucleic acid) along an electric field (e.g., in the direction of current), based on a charge of the molecule. Electrophoresis can induce electrotransfer, for example, by moving a molecule (e.g., a nucleic acid, e.g., a naked nucleic acid) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid, e.g., a naked nucleic acid) from outside the target cell to inside the target cell. Thus, in some instances, electrotransfer occurs as a result of a combination of electrophoresis and electroporation.

As used herein, the term “circular DNA vector” refers to a DNA molecule in a circular form. Such circular form is typically capable of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector,” “c3DNA,” and “C³DNA.” A skilled artisan will understand that such circular vectors include vectors that are covalently closed with supercoiling and complex DNA topology, as is described herein. In particular embodiments, a circular DNA vector is supercoiled (e.g., monomeric supercoiled). In certain instances, a circular DNA vector lacks a bacterial origin of replication.

As used herein, the term “therapeutic sequence” refers to the portion of a DNA molecule that contains any genetic material required for transcription in a target cell of one or more therapeutic moieties, which may include one or more coding sequences, promoters, terminators, introns, and/or other regulatory elements. A therapeutic moiety can be a therapeutic protein (e.g., a replacement protein (e.g., a protein that replaces a defective protein in the target cell) or an endogenous protein) and/or a therapeutic nucleic acid (e.g., one or more microRNAs or CRISPR molecules). In DNA vectors having more than one transcription unit, the therapeutic sequence contains the plurality of transcription units. A therapeutic sequence may include one or more genes (e.g., heterologous genes or transgenes) to be administered for a therapeutic purpose.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides (e.g., polypeptides), native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject (e.g., cystic fibrosis transmembrane regulator (CFTR)).

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors.

As used herein, the term “therapeutic nucleic acid” refers to a nucleic acid that binds to (e.g., hybridizes with) a molecule (e.g., protein or nucleic acid) in the subject to confer its therapeutic effect (i.e., without necessarily being transcribed or translated). Therapeutic nucleic acids can be DNA or RNA, such as small interfering RNA (siRNA), short hairpin RNA (shRNA), microRNA (miRNA), a CRISPR molecule (e.g., guide RNA (gRNA) (e.g., a gRNA/Cas9 or gRNA/Cas12 RNP)), an oligonucleotide (e.g., an antisense oligonucleotide), an aptamer, or a DNA vaccine. In some embodiments, the therapeutic nucleic acid may be a non-inflammatory or a non-immunogenic therapeutic nucleic acid. In other embodiments, the therapeutic nucleic acid is recognizable by the immune system (e.g., adaptive immune system) and may induce an immune response (e.g., an innate immune response). Such therapeutic nucleic acids include toll-like receptor (TLR) agonists.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, a “vector” refers to a nucleic acid molecule capable of carrying a sequence of interest (e.g., a therapeutic sequence) to which is it linked into a target cell in which the sequence of interest can then be transcribed, replicated, processed, and/or expressed in the target cell. After a target cell or host cell processes the sequence of interest (e.g., a therapeutic sequence) of the vector, the sequence of interest (e.g., a therapeutic sequence) is not considered a vector. One type of vector is a “plasmid”, which refers to a circular double stranded DNA loop containing a bacterial backbone into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”).

As used herein, the term “individual” includes any mammal in need of treatment or prophylaxis, e.g., by a synthetic circular DNA vector described herein. In some embodiments, the individual or subject is a human. In other embodiments, the individual or subject is a non-human mammal (e.g., a non-human primate (e.g., a monkey), a mouse, a pig, a rabbit, a cat, or a dog). The individual or subject may be male or female.

As used herein, an “effective amount” or “effective dose” of a therapeutic circular DNA vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, therapeutic circular DNA vectors of the invention are used to delay development of a disease or to slow the progression of a disease.

The terms “level of expression” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a sequence of interest (e.g., a therapeutic sequence), or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A sequence of interest (e.g., a therapeutic sequence), or functional portion thereof, may be expressible if it is not silenced, e.g., by DNA methylation and/or histone methylation and compaction. Expression persistence can be assessed by detecting or quantifying (i) mRNA transcribed from the sequence of interest (e.g., a therapeutic sequence) in the target cell or progeny thereof (e.g., through qPCR, RNA-seq, or any other suitable method) and (ii) protein translated from the sequence of interest (e.g., a therapeutic sequence) in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the sequence of interest (e.g., a therapeutic sequence) in the target cell or progeny thereof and (ii) protein translated from the sequence of interest (e.g., a therapeutic sequence) in the target cell or progeny thereof. Expression persistence of a sequence of interest (e.g., a therapeutic sequence), or a functional portion thereof, can be quantified relative to a reference vector, such as a control vector produced in bacteria (e.g., a circular vector produced in bacteria or having one or more bacterial signatures not present in the vector of the invention (e.g., a plasmid)), using any gene expression characterization method known in the art. Expression persistence can be quantified at any given time point following administration of the vector. For example, in some embodiments, expression of a therapeutic circular DNA vector of the invention persists for at least two weeks after administration if it is detectable in the target cell, or progeny thereof, two weeks after administration of the therapeutic circular DNA vector. In some embodiments, expression of a gene “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a sequence of interest (e.g., a therapeutic sequence) is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, the term “cystic fibrosis transmembrane regulator (CFTR)” refers to any native CFTR from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known CFTR signaling (e.g., ion and water secretion and absorption in epithelial tissues). CFTR encompasses full-length, unprocessed CFTR, as well as any form of CFTR that results from native processing in the cell. An exemplary human CFTR sequence is provided as National Center for Biotechnology Information (NCBI) Gene ID: 1080.

The terms “a” and “an” mean “one or more of” For example, “a gene” is understood to represent one or more such genes. As such, the terms “a” and “an,” “one or more of a (or an),” and “at least one of a (or an)” are used interchangeably herein.

As used herein, the term “about” refers to a value within ±10% variability from the reference value, unless otherwise specified.

### II. Methods

Provided herein are methods of expressing a sequence of interest (such as a therapeutic sequence) in a target airway cell. In some instances, the method includes the following steps: (i) exposing the target airway cell to a vector (e.g., non-integrating vectors, such as a synthetic circular DNA vector, or integrating vectors, such as CRISPR vectors) comprising the sequence of interest (e.g., a therapeutic sequence), wherein the target airway cell is in electrical communication with an electrode; and (ii) transmitting one or more pulses of electrical energy through the electrode at conditions suitable for electrotransfer of the synthetic circular DNA vector into the target airway cell, thereby expressing the sequence of interest (e.g., a therapeutic sequence). In some instances, the target airway cell is an airway epithelial cell. In some embodiments, the target airway cell is a lung cell. In some embodiments, the target airway cell is a nasal epithelial cell, a tracheal cell, bronchial cell, bronchiolar cell, or an alveolar cell.

Step (ii) above may be performed in situ in an individual by inserting a catheter comprising the electrode into an airway lumen of the individual. In some embodiments, the airway lumen is trachea, bronchi, or bronchiole. In some embodiments, the individual has been diagnosed with a disease or disorder. In some embodiments, the disease or disorder is a monogenic respiratory disease, e.g., cystic fibrosis.

Synthetic circular DNA vectors can be administered to the individual locally. In some embodiments, the local administration is intranasal administration or intramuscular administration. In some embodiments, the local administration is by flood, spray, or aerosolization of the synthetic circular DNA vector. For administration by inhalation, the administering solution can be conveniently delivered in the form of an aerosol spray from a pressurized pack or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of gelatin, for example, for use in an inhaler or insufflator, can be formulated containing a powder mix of the molecule of interest and a suitable powder base such as lactose or starch.

Alternatively, synthetic circular DNA vectors can be administered to the individual systemically, e.g., by intravenous administration.

In some instances, the method further includes administering the synthetic circular DNA vector. In some embodiments, the synthetic circular DNA vector is administered to the individual locally. In some embodiments, the local administration is intranasal administration or intramuscular administration. In some embodiments, the local administration is by flood, spray, or aerosolization of the synthetic circular DNA vector. In some embodiments, the synthetic circular DNA vector is administered to the individual systemically. In some embodiments, the systemic administration is intravenous administration. In some embodiments, partial airway epithelial ablation therapy has preceded the administration of the synthetic circular DNA vector. In some embodiments, the partial airway epithelial ablation was administered using a pulsed electric field. In some embodiments, the method further comprises administering the partial airway epithelial ablation therapy.

In another aspect, the invention provides a method of delivering a therapeutic agent (e.g., a therapeutic sequence encoding a protein (e.g., as part of a synthetic circular DNA vector) or a therapeutic nucleic acid (e.g., a CRISPR molecule)) to a target airway cell in an individual, the method including: (i) inserting a catheter comprising a monopolar electrode into an airway lumen of the individual; (ii) positioning the electrode within a target region of the airway lumen comprising the target airway cell and the therapeutic agent; (iii) while the electrode is within the target region, transmitting one or more pulses of electrical energy through the electrode at conditions suitable for electrotransfer of the therapeutic agent into the target airway cell. In some embodiments, the individual has been treated with a partial airway epithelial ablation therapy.

In another aspect, the invention provides a method of delivering a synthetic circular DNA vector to a target airway cell in an individual, the method including: (i) inserting a catheter comprising a monopolar electrode into an airway lumen of the individual; (ii) positioning the electrode within a target region of the airway lumen comprising the target airway cell and the therapeutic agent; (iii) while the electrode is within the target region, transmitting one or more pulses of electrical energy through the electrode at conditions suitable for electrotransfer of the therapeutic agent into the target airway cell. In some embodiments, the individual has been treated with a partial airway epithelial ablation therapy.

In another aspect, the invention provides a method of delivering a synthetic circular DNA vector to a target airway cell in an individual that has been treated with a partial airway epithelial ablation therapy, the method comprising: (i) inserting a catheter comprising an electrode into an airway lumen of the individual; (ii) positioning the electrode within a target region of the airway lumen comprising the target airway cell and the therapeutic agent; and (iii) while the electrode is within the target region, transmitting one or more pulses of electrical energy through the electrode at conditions suitable for electrotransfer of the therapeutic agent into the target airway cell.

In another aspect, the invention provides a method of delivering a therapeutic agent (e.g., a therapeutic sequence encoding a protein (e.g., as part of a synthetic circular DNA vector) or a therapeutic nucleic acid (e.g., a CRISPR molecule)) to a target airway cell in an individual that has been treated with a partial airway epithelial ablation therapy, the method comprising: (i) inserting a catheter comprising an electrode into an airway lumen of the individual; (ii) positioning the electrode within a target region of the airway lumen comprising the target airway cell and the therapeutic agent; and (iii) while the electrode is within the target region, transmitting one or more pulses of electrical energy through the electrode at conditions suitable for electrotransfer of the therapeutic agent into the target airway cell. In another aspect, the invention provides a method of treating a disease or disorder in an individual, the method comprising: (i) positioning an electrode in electrical communication with target airway cells of the individual, wherein the target airway cells are exposed to a therapeutically effective amount of a synthetic circular DNA vector comprising a therapeutic sequence; and (ii) transmitting one or more pulses of electrical energy through the electrode at conditions suitable for electrotransfer of the synthetic circular DNA vector into the target airway cells, thereby expressing the therapeutic sequence and treating the disease or disorder.

Contemplated herein are methods involving homology directed repair of disease-associated gene mutations (e.g., the W1282X mutation in CFTR for cystic fibrosis treatment) by delivery of CRISPR molecules including use of known Cas9 and Cas12a CRISPR editing methods. Thus, some embodiments of the methods described herein include delivery of Cas9/gRNA or Cas12a/gRNA ribonucleoproteins (RNPs) and single strand DNA (ssODN) oligonucleotide donors. Such CRISPR methods are described in Santos et al., J. Cyst. Fibros. 2022 January: 21(1):181-187, which is incorporated herein by reference. Some embodiments of the methods described herein include delivery of nucleic acids encoding CRISPR molecules, including nucleic acids encoding Cas9, Cas12a, one or more gRNAs, or combinations thereof.

A vector (e.g., a synthetic circular DNA vector or a therapeutic nucleic acid (e.g., a CRISPR molecule)) can be administered within 24 hours of transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds preceding transmission of an electric field).

Conditions suitable for electrotransfer of the therapeutic agent into the target airway cell comprise a voltage at the target lung cell of less than 2,400 V. In some embodiments, the voltage at the target airway cell is between 10 V and 2,400 V, e.g., between 100 V and 2,400 V, e.g., from 100 V to 500 V, from 500 V to 1,000 V, from 1,000 V to 1,500 V, or from 1,500 V to 2,000 V. In some embodiments, the voltage at the target airway cell is between 1600 V and 2,400 V, between 1800 V and 2,400 V, or between 2,000 V and 2,400 V). In some embodiments, 1-10 (e.g., 1-6) pulses of electrical energy are transmitted. In some embodiments, the total number of pulses of electrical energy are transmitted within 1-20 seconds. In some embodiments, a single pulse (i.e., one and only one pulse) of electrical energy is transmitted. In other embodiments, exactly two pulses of electrical energy is transmitted. In some embodiments, the one or more pulses of electrical energy are square waveforms. In some embodiments, the one or more pulses of electrical energy have an amplitude from 10 V to 10,000 V, e.g., from 100 V to 5,000 V, e.g., from 100 V to 500 V, from 500 V to 1,000 V, from 1,000 V to 1,500 V, or from 1,500 V to 2,000 V (e.g., from 1,600 V to 2,000 V, from 1,600 V to 1,800 V, or from 1,800 V to 2,000 V). In some embodiments, each of the pulses of electrical energy is from 0.01 to 200 milliseconds in duration. In some embodiments, each of the pulses of electrical energy is from 10 to 50 milliseconds in duration (e.g., about 20 milliseconds in duration). In some embodiments, the total duration of all of the pulses of electrical energy is from 10 to 50 milliseconds (e.g., about 20 milliseconds in total duration). In some embodiments, the electrode is a monopolar electrode. In other embodiments, the electrode is part of a bipolar electrode configuration. In some embodiments, the electrode is within 10 cm of the target airway cell. For example, in some embodiments, the electrode is within 1 cm of the target airway cell (e.g., within 5 mm, within 4 mm, or within 3 mm).

Catheter based electrodes useful as part of such methods include those described in, e.g., International Patent Publication No. WO 2018/005511 and WO 2019/133606, which are herein incorporated by reference in their entirety.

### III. Synthetic Circular DNA Vectors

Synthetic circular DNA vectors persist intracellularly (e.g., in dividing or in quiescent cells, such as post-mitotic cells) as episomes, e.g., in a manner similar to AAV vectors. In any of the embodiments, described herein, a synthetic circular DNA vector may be a non-integrating vector. Synthetic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG islands or CpG motifs)) or components additionally, or otherwise associated with reduced persistence (e.g., CpG islands or CpG motifs). The synthetic circular DNA vectors produced as described herein feature one or more therapeutic sequences and may lack plasmid backbone elements (e.g., bacterial elements such as (i) a bacterial origin of replication and/or (ii) a drug resistance gene) and a recombination site.

Synthetic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG motifs)) or components additionally or otherwise associated with reduced persistence (e.g., CpG islands). For example, in some embodiments, the vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dcm methylation. For examples, in some embodiments, the vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dcm methylase).

In some embodiments, the synthetic circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector produced in bacteria or having one or more bacterial signatures not present in the vector of the invention, e.g., plasmid DNA). In some embodiments, expression persistence of the synthetic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, intra-cellular persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, therapeutic persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, the reference vector is a circular vector or plasmid that (a) has the same therapeutic sequence as a therapeutic circular DNA vector to which it is being compared, and (b) is produced in bacteria and/or has one or more bacterial signatures not present in the therapeutic circular DNA vector to which it is being compared, which signatures may include, for example, an antibiotic resistance gene or a bacterial origin of replication.

In some embodiments, expression of a synthetic circular DNA vector persists for one week, two weeks, three weeks, four weeks, six weeks, two months, three months, four months, five months, six months, seven months, eight months, nine months, ten months, eleven months, one year, or longer after administration.

In some embodiments, expression and/or therapeutic effect of the synthetic circular DNA vector persists for one week to four weeks, from one month to four months, or from four months to one year (e.g., at least one week, at least two weeks, at least one month, or longer). In some embodiments, the expression level of the synthetic circular DNA vector does not decrease by more than 90%, by more than 50%, or by more than 10% in the 1 week or more, e.g., 2 weeks, 3 weeks, 5 weeks, 7 weeks, 9 weeks or more, 13 weeks or more, 18 weeks or more following transfection from levels observed within the first 1, 2, or 3 days.

The synthetic circular DNA vector may be monomeric, dimeric, trimeric, tetrameric, pentameric, hexameric, etc. In some preferred embodiments, the circular DNA vector is monomeric. In some embodiments, the DNA vector is supercoiled. In some embodiments, the therapeutic circular DNA vector is a monomeric, supercoiled circular DNA molecule. In some embodiments, the therapeutic circular DNA vector is nicked. In some embodiments, the therapeutic circular DNA vector is open circular. In some embodiments, the therapeutic circular DNA vector is double-stranded circular.

Synthetic circular DNA vectors described herein contain a therapeutic sequence, which may include one or more protein-coding domain and/or one or more non-protein coding domains (e.g., a therapeutic nucleic acid).

In particular embodiments involving a therapeutic protein-coding therapeutic domain, the therapeutic sequence includes, linked in the 5′ to 3′ direction: a promoter and a single therapeutic protein-coding domain (e.g., a single transcription unit); a promoter and two or more therapeutic protein-coding domains (e.g., a multicistronic unit); or a first transcription unit and one or more additional transcription units (e.g., a multi-transcription unit). Any such protein-coding therapeutic sequences may further include non-protein coding domains, such as polyadenylation sites, control elements, enhancers, sequences to mark DNA (e.g., for antibody recognition), PCR amplification sites, sequences that define restriction enzyme sites, site-specific recombinase recognition sites, sequences that are recognized by a protein that binds to and/or modifies nucleic acids, linkers, splice sites, pre-mRNA binding domains, regulatory sequences, and/or a therapeutic nucleic acid (e.g., a microRNA-encoding sequence). Therapeutic protein-coding domains can be full-length protein-coding domains (e.g., corresponding to a native gene or natural variant thereof) or a functional portion thereof, such as a truncated protein-coding domain (e.g., minigene).

In some embodiments, the therapeutic sequence encodes a monomeric protein (e.g., a monomeric protein with secondary structure under physiological conditions, e.g., a monomeric protein with secondary and tertiary structure under physiological conditions, e.g., a monomeric protein with secondary, tertiary, and quaternary structure under physiological conditions). Additionally, or alternatively, the therapeutic sequence may encode a multimeric protein (e.g., a dimeric protein (e.g., a homodimeric protein or heterodimeric protein), a trimeric protein, etc.)

In some embodiments, the therapeutic sequence encodes cystic fibrosis transmembrane regulator.

In some embodiments, the therapeutic sequence is from 0.1 Kb to 100 Kb in length (e.g., the therapeutic gene sequence is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic sequence is at least 10 Kb (e.g., from 10 Kb to 15 Kb, from 15 Kb to 20 Kb, or from 20 Kb to 30 Kb; e.g., from 10 Kb to 13 Kb, from 10 Kb to 12 Kb, or from 10 Kb to 11 Kb; e.g., from 10-11 Kb, from 11-12 Kb, from 12-13 Kb, from 13-14 Kb, or from 14-15 Kb). In some embodiments, the therapeutic sequence is at least 1,100 bp in length (e.g., from 1,100 bp to 10,000 bp, from 1,100 bp to 8,000 bp, or from 1,100 bp to 5,000 bp in length). In some embodiments, the therapeutic sequence is at least 2,500 bp in length (e.g., from 2,500 bp to 15,000 bp, from 2,500 bp to 10,000 bp, or from 2,500 bp to 5,000 bp in length; e.g., from 2,500 bp to 5,000 bp, from 5,000 bp to 7,500 bp, from 7,500 bp to 10,000 bp, from 10,000 bp to 12,500 bp, or from 12,500 bp to 15,000 bp). In some embodiments, the therapeutic sequence is at least 8,000 bp, at least 9,000 bp, at least 10,000 bp, at least 11,000 bp, at least 12,000 bp at least 13,000 bp, at least 14,000 bp, at least 15,000 bp, at least 16,000 bp (e.g., 11,000 bp to 16,000 bp, 12,000 bp to 16,000 bp, 13,000 bp to 16,000 bp, 14,000 bp to 16,000 bp, or 15,000 bp to 16,000 bp). In particular embodiments, the therapeutic sequence is sufficiently large to encode a protein and is not an oligonucleotide therapy (e.g., not an antisense, siRNA, shRNA therapy, etc.).

In some embodiments, the 3′ end of the therapeutic sequence is connected to the 5′ end of the therapeutic sequence in a therapeutic circular DNA vector by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, or 8 bp). For example, in any of the therapeutic circular DNA vectors generated using type IIs restriction enzymes, the 3′ end of the therapeutic sequence may be connected to the 5′ end of the therapeutic sequence by a non-bacterial sequence corresponding to sticky end or overhang of the type IIs restriction enzyme cut site (e.g., TTTT or AAAA).

In some embodiments, the therapeutic sequence includes a reporter sequence in addition to a therapeutic protein-encoding domain or a therapeutic non-protein encoding domain. Such reporter genes can be useful in verifying therapeutic gene sequence expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

In some embodiments, the therapeutic sequence lacks a reporter sequence.

As part of the therapeutic sequence, therapeutic circular DNA vectors of the invention may include conventional control elements which modulate or improve transcription, translation, and/or expression in a target cell. Suitable control elements are described in International Publication No. WO 2021/055760, which is incorporated herein by reference in its entirety.

### IV. Pharmaceutical Compositions

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂, CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂) or potassium chloride (KCl), wherein further anions may be present. CaCl₂ can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

In some instances, synthetic circular DNA vectors, and compositions thereof, are administered locally to the airway, e.g., through aerosols or nebulization.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

Synthetic circular DNA vectors according to the invention may be administered naked in a suitable buffer without being associated with any further vehicle, transfection, or complexation agent.

In certain embodiments of the invention, any of the therapeutic circular DNA vectors of the invention can be complexed with one or more cationic or polycationic compounds, e.g., cationic or polycationic polymers, cationic or polycationic peptides or proteins, e.g., protamine, cationic or polycationic polysaccharides, and/or cationic or polycationic lipids.

According to a particular embodiment, the therapeutic circular DNA vector of the invention may be complexed with lipids to form one or more liposomes, lipoplexes, or lipid nanoparticles. Therefore, in one embodiment, the pharmaceutical composition comprises liposomes, lipoplexes, and/or lipid nanoparticles comprising a therapeutic circular DNA vector. Such lipid-based delivery systems may be formulated for pulmonary delivery according to any suitable method known in the art or described herein.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for therapeutic circular DNA vectors. Cationic lipids, such as MAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Lipid nanoparticle (LNP) compositions and other lipid-based carriers shown to be suitable for delivery to the airway (e.g., by aerosol exposure) are contemplated as part of the present invention. For instance, any of the LNP compositions described in International Publication No.: WO 2022/104131, which is hereby incorporated by reference in its entirety, can be used to encapsulate any of the expression constructs or nucleic acid vectors described herein, e.g., for delivery to the target airway tissue, e.g., as an aerosolizable composition. Other known aerosolizable and/or nebulizable lipid compositions (e.g., lipid-nanovesicles) contemplated herein as adaptable for encapsulation and delivery of the nucleic acid vectors described herein include those described in, e.g., Kaur et al., Front Pharmacol. 2022 March 10; 12:734913; Vartak et al, Nanomedicine, 2021 June; 16(14):1187-1202; Xu et al., Int. J. Nanomedicine, 2021 Feb. 16; 16:1221-1229; and Elhissi, Curr. Pharm. Des. 2017; 23(3):362-372.

Thus, in one embodiment of the invention, the therapeutic circular DNA vector of the invention is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition according to the invention comprises the therapeutic circular DNA vector of the invention that is formulated together with a cationic or polycationic compound and/or with a polymeric carrier. Accordingly, in a further embodiment of the invention, the therapeutic circular DNA vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the therapeutic circular DNA vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the therapeutic circular DNA vector according to the invention is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g., selected from a cationic polymer as mentioned above) and of one or more hydrophilic or hydrophobic blocks (e.g. polyethyleneglycole); etc.

According to a particular embodiment, the pharmaceutical composition of the invention includes the therapeutic circular DNA vector encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the therapeutic circular DNA vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the therapeutic circular DNA vector of the present invention may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

Any of the compositions described herein may be formulated in a suitable form for pulmonary administration, e.g., via the buccal cavity. Such a formulations may include dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50% to 99.9% (w/w) of the composition, and active ingredient may constitute 0.1%>to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Pharmaceutical compositions described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm.

Pharmaceutical compositions suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### EXAMPLES

### Example 1. Comparison of Electrotransfer-Mediated Expression by Synthetic Circular DNA Vectors with that of Plasmid DNA Vectors in Human Bronchial Epithelial Cells

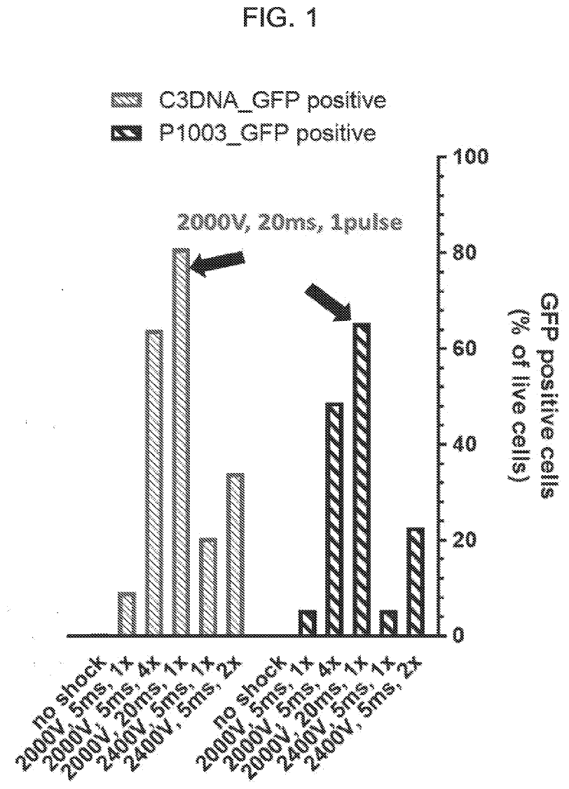

Primary human bronchial epithelial cells were suspended in culture media containing either synthetic circular DNA vector (c3DNA) encoding GFP or an equimolar amount of plasmid DNA vector (P1003) encoding GFP. Pulsed electric fields were applied at the conditions indicated in FIG. 1 (no shock; 2000 V/5 ms/1 pulse; 2000 V/5 ms/4 pulses; 2000 V/20 ms/1 pulse; 2400 V/5 ms/1 pulse; and 2400 V/5 ms/2 pulses) using the Neon Transfection System (Thermo Fisher Scientific). Cells were plated in wells after electroporation. GFP expression was assessed with flow cytometry 48 hours after transfection. GFP expression level was measured as a percent of live cells.

Synthetic circular DNA vectors showed greater expression levels of GFP compared to plasmid DNA vectors. Electroporation using conditions of 2000 V, 20 ms, 1 pulse showed the highest transfection efficiency in both synthetic circular DNA vectors and plasmid DNA vectors (FIG. 1). Additionally, increasing the pulse number at higher voltages, regardless of pulse length, decreased viability by ˜50%.

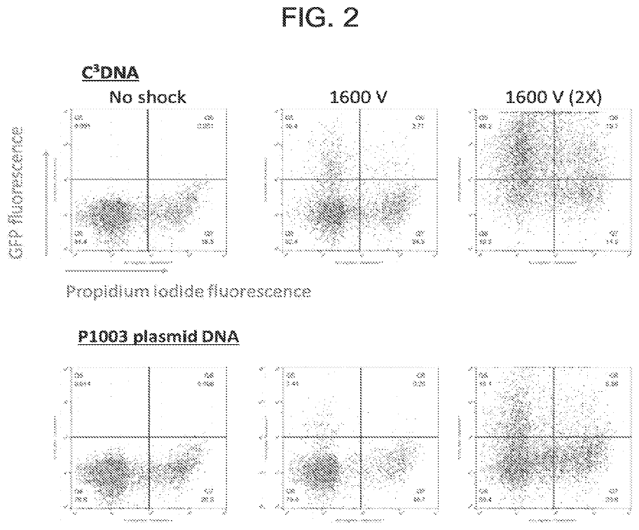

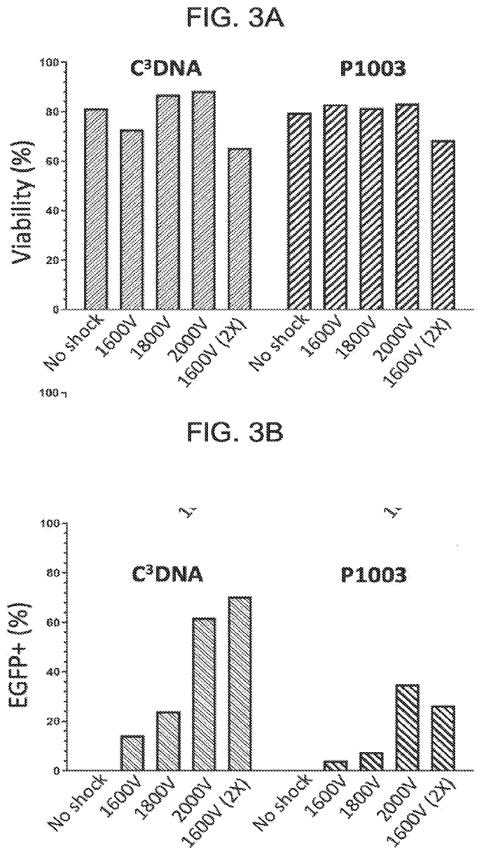

In a second experiment, pulsed electric fields were applied across a lower range of voltages, 1600 V, 1800 V, 2000 V, or 1600 V (x2), and cells were collected for transfection and viability analysis by flow cytometry 72 hours after electroporation. Exemplary gating strategies and results are shown in FIG. 2. Viability was similarly retained across all conditions for both C³DNA and plasmid (FIGS. 2 and 3A); however, C³DNA conferred greater transgene expression than plasmid in all conditions (FIGS. 2 and 3B).

### Example 2. Electrotransfer of Synthetic Circular DNA Vector in Air-Liquid Interface (ALI) Cultures of Well Differentiated Human Bronchial Epithelium

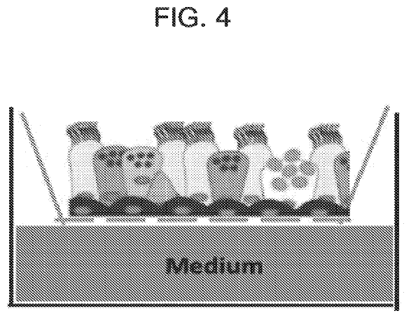

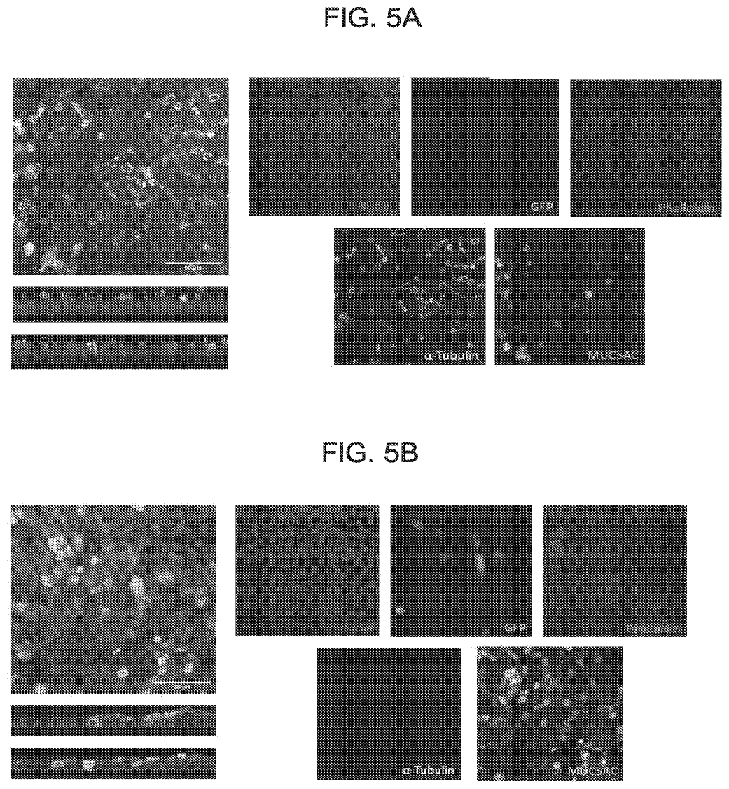

Human ALI cultures were prepared according to known methods as shown in FIG. 4 and verified as well differentiated by expression of a-tubulin (for ciliated cells) and muc5AC (for goblet cells) (FIGS. 5A and 5B). ALI cultures were incubated with 6 μg of synthetic circular DNA vector encoding GFP for 10 minutes.

Pulsed electric field was applied across the membrane using a bipolar electrode configuration that generates an electric field perpendicular to the ALI culture, in which (a) a first electrode was placed beneath the culture such that its surface was facing up, parallel with the cell layer; and (b) a second electrode was place above the culture such that its surface was facing down, parallel to the cell layer. The distance between electrodes was 3 mm.

72 hours after transfection, cells were visualized for GFP expression. In the control group, which was not exposed to pulsed electric field, cultures had normal morphology, but no GFP was observed (FIG. 5A). In contrast, the experimental group, which was exposed to two 5 ms pulses of 250 V exhibited significant GFP expression in epithelial cells (FIG. 5B). As shown in the X-Z confocal images (bottom left panels of FIG. 5B), transfection was observed in cells with a luminal border, which are thought to be essential for transmembrane function, e.g., CFTR function. Ciliated cells were ablated by the PEF (as indicated by the lack of acetylated tubulin staining), leaving goblet cells and stem cells (FIG. 5B).

### Example 3. Electrotransfer of Synthetic Circular DNA Vector in Tracheal Explant Cultures

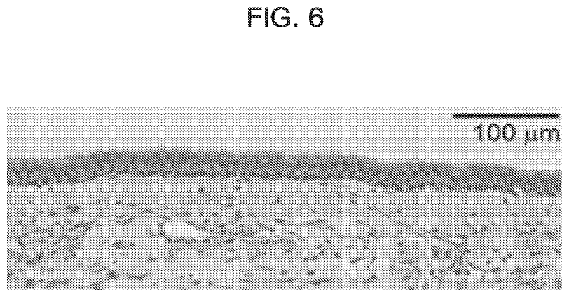

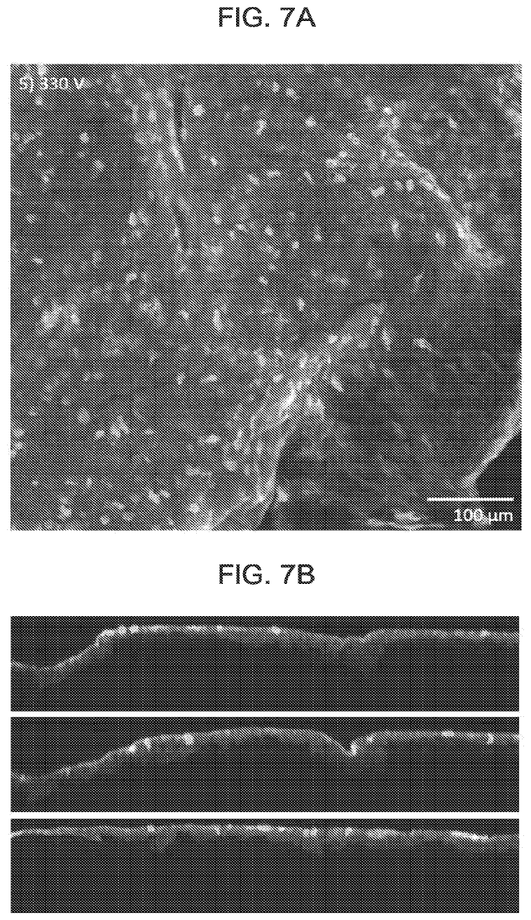

Rabbit tracheal explants were provided (a section of which, stained with H&E, is shown in FIG. 6). Cultures were exposed to 12 μg of synthetic circular DNA vector encoding GFP (in 2 mL volume) for 10 minutes. Pulsed electric field was applied as described in Example 2, except pulse amplitude was 330 V. 72 hours after transfection, cells were visualized for GFP expression. A top view (FIG. 7A) and side view (FIG. 7B) of the epithelium show that GFP was observed in cells throughout the epithelium.

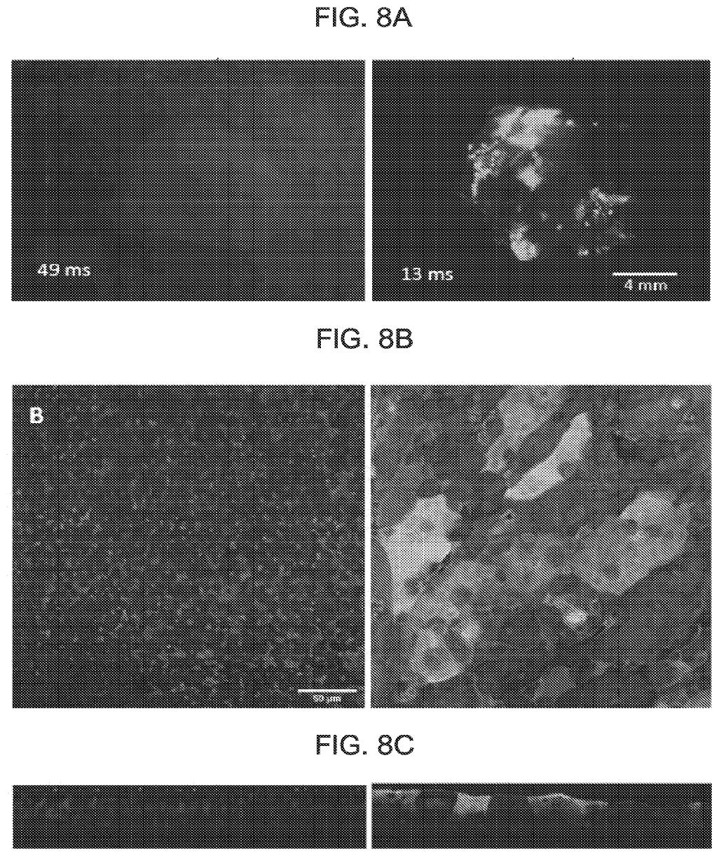

In a follow-on experiment, explant cultures were exposed to 50 μg of synthetic circular DNA vector encoding GFP in 0.8 mL volume for 10 minutes. Pulsed electric field was applied using an electrode system in which electrodes were separated by a distance 4 mm, with the explant positioned therebetween. Four five-millisecond pulses at 300 V were administered. Pulsed electric field-treated explants were compared to non-pulsed electric field-treated explants as shown in FIGS. 8A (showing live cell fluorescence imaging of a single cell), FIG. 8B (showing X-Y confocal images), and FIG. 8C (showing X-Z confocal images), which confirm luminal cell expression of GFP in explants treated with pulsed electric field, whereas none was observed in untreated explants.

### Example 4. Expression of a CFTR in Lung Epithelium In Situ

The patient is a cystic fibrosis patient having a DeltaF508 mutation in CFTR. A physician first administers a partial airway epithelial ablation therapy using a clinically available transbronchoscopic pulsed electric field system to remove columnar cells, thereby exposing basal stem cells in the bronchi.

A synthetic circular DNA vector containing a functional CFTR gene is synthesized using a cell-free method adapted from the method described in International Patent Publication No. WO 2021/055760. The synthetic circular DNA vector is formulated in a nebulizer with dichlorodifluoromethane, trichlorofluoromethane, dichlorotetrafluoroethane, carbon dioxide or other suitable gas and administered by inhalation to the patient.

Within five to thirty minutes from inhalation of the synthetic circular DNA vector encoding CFTR, a catheter having an electrode on its distal terminus is progressed through the patient's airway by a physician. The physician positions the electrode in the bronchial lumen and administers two 20 ms pulses of amplitude 250 V. The electrode is removed from the patient, and the procedure is complete. After the procedure, the patient is monitored weekly to assess disease progression.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the following numerated embodiments and claims.

### Numerated Embodiments

1. A method of expressing a therapeutic sequence in a target airway cell, the method comprising:
(i) exposing the target airway cell to a synthetic circular DNA vector comprising the therapeutic sequence, wherein the target airway cell is in electrical communication with an electrode; and
(ii) transmitting one or more pulses of electrical energy through the electrode at conditions suitable for electrotransfer of the synthetic circular DNA vector into the target airway cell, thereby expressing the therapeutic sequence.

2. The method of embodiment 1, wherein the synthetic circular DNA vector comprises a therapeutic sequence having a 5′ end and a 3′ end, wherein the 5′ end is connected to the 3′ end by a non-bacterial sequence.

3. The method of embodiment 2, wherein the non-bacterial sequence corresponds to a restriction enzyme cut site overhang.

4. The method of any one of embodiments 1-3, wherein the synthetic circular DNA vector is a naked synthetic circular DNA vector.

5. The method of any one of embodiments 1-4, wherein the synthetic circular DNA vector is encapsulated in, or associated with, a particulate structure.

6. The method of embodiment 5, wherein the particulate structure is a liposome.

7. The method of any one of embodiments 1-6, wherein the therapeutic sequence comprises a therapeutic protein-encoding sequence.

8. The method of embodiment 7, wherein the therapeutic protein is cystic fibrosis transmembrane receptor (CFTR).

9. The method of any one of embodiments 1-8, wherein the therapeutic sequence comprises one or more regulatory elements.

10. The method of any one of embodiments 1-6, wherein the therapeutic sequence comprises a therapeutic nucleic acid.

11. The method of any one of embodiments 1-10, wherein the target airway cell is an airway epithelial cell.

12. The method of any one of embodiments 1-11, wherein the target airway cell is a lung cell.

13. The method of any one of embodiments 1-12, wherein the target airway cell is a nasal epithelial cell, a tracheal cell, bronchial cell, bronchiolar cell, or an alveolar cell.

14. The method of any one of embodiments 1-13, wherein the conditions suitable for electrotransfer of the therapeutic agent into the target airway cell comprise a voltage at the target lung cell of less than 2,400 V.

15. The method of embodiment 14, wherein the voltage at the target airway cell is between 100 V and 2,400 V.

16. The method of any one of embodiments 1-15, wherein 1-6 pulses of electrical energy are transmitted.

17. The method of any one of embodiments 1-16, wherein the total number of pulses of electrical energy are transmitted within 1-20 seconds.

18. The method of embodiment 16, wherein a single pulse of electrical energy is transmitted.

19. The method of any one of embodiments 1-18, wherein the one or more pulses of electrical energy are square waveforms.

20. The method of any one of embodiments 1-19, wherein the one or more pulses of electrical energy have an amplitude from 100 V to 5,000 V.

21. The method of any one of embodiments 1-20, wherein each of the pulses of electrical energy is from 0.01 to 200 milliseconds in duration.

22. The method of embodiment 21, wherein each of the pulses of electrical energy is from 10 to 50 milliseconds in duration.

23. The method of embodiment 22, wherein the total duration of all of the pulses of electrical energy is from 10 to 50 milliseconds.

24. The method of any one of embodiments 1-23, wherein the electrode is a monopolar electrode.

25. The method of any one of embodiments 1-23, wherein the electrode is part of a bipolar electrode configuration.

26. The method of any one of embodiments 1-25, wherein the electrode is within 10 cm of the target airway cell.

27. The method of embodiment 26, wherein the electrode is within 1 cm of the target airway cell.

28. The method of any one of embodiments 1-27, wherein the expression level of the therapeutic sequence from the synthetic circular DNA vector is greater than expression level of a therapeutic sequence from a reference plasmid DNA vector under analogous conditions.

29. The method of embodiment 28, wherein the expression level of the therapeutic sequence from the synthetic circular DNA vector is at least 10% greater than expression level of a therapeutic sequence from a reference plasmid DNA vector under analogous conditions.

30. The method of any one of embodiments 1-29, wherein the expression persistence of the therapeutic sequence from the synthetic circular DNA vector is greater than expression persistence of a therapeutic sequence from a reference plasmid DNA vector under analogous conditions.

31. The method of any one of embodiments 1-30, wherein the target airway cell has a mutation in a gene correctable by the therapeutic sequence.

32. The method of any one of embodiments 1-31, wherein step (ii) is performed in situ in an individual by inserting a catheter comprising the electrode into an airway lumen of the individual.

33. The method of embodiment 32, wherein the airway lumen is trachea, bronchi, or bronchiole.

34. The method of embodiment 32 or 33, wherein the individual has been diagnosed with a disease or disorder.

35. The method of embodiment 34, wherein the disease or disorder is a monogenic respiratory disease.

36. The method of embodiment 35, wherein the monogenic respiratory disease is cystic fibrosis.

37. The method of any one of embodiments 1-36, wherein the synthetic circular DNA vector has been administered to the individual locally.

38. The method of embodiment 37, wherein the local administration was intranasal administration or intramuscular administration.

39. The method of embodiment 37, wherein the local administration was by flood, spray, or aerosolization of the synthetic circular DNA vector.

40. The method of any one of embodiments 1-36, wherein the synthetic circular DNA vector has been administered to the individual systemically.

41. The method of embodiment 40, wherein the systemic administration was intravenous administration.

42. The method of any one of embodiments 1-41, wherein the method further comprises administering the synthetic circular DNA vector.

43. The method of any one of embodiments 1-42, wherein partial airway epithelial ablation therapy has preceded the administration of the synthetic circular DNA vector.

44. The method of embodiment 43, wherein the partial airway epithelial ablation was administered using a pulsed electric field.

45. The method of any one of embodiments 1-44, wherein the method further comprises administering the partial airway epithelial ablation therapy.

46. The method of any one of embodiments 1-45, wherein the target airway cell is a human cell.

47. A method of delivering a therapeutic agent to a target airway cell in an individual, the method comprising:
(i) inserting a catheter comprising a monopolar electrode into an airway lumen of the individual;
(ii) positioning the electrode within a target region of the airway lumen comprising the target airway cell and the therapeutic agent;
(iii) while the electrode is within the target region, transmitting one or more pulses of electrical energy through the electrode at conditions suitable for electrotransfer of the therapeutic agent into the target airway cell.

48. The method of embodiment 47, wherein the individual has been treated with a partial airway epithelial ablation therapy.

49. A method of delivering a therapeutic agent to a target airway cell in an individual that has been treated with a partial airway epithelial ablation therapy, the method comprising:
(i) inserting a catheter comprising an electrode into an airway lumen of the individual;
(ii) positioning the electrode within a target region of the airway lumen comprising the target airway cell and the therapeutic agent; and
(iii) while the electrode is within the target region, transmitting one or more pulses of electrical energy through the electrode at conditions suitable for electrotransfer of the therapeutic agent into the target airway cell.

50. The method of any one of embodiments 47-49, wherein the therapeutic agent comprises a nucleic acid vector comprising a therapeutic sequence.

51. The method of any one of embodiments 47-50, wherein the nucleic acid vector is a non-viral nucleic acid vector.

52. The method of any one of embodiments 47-51, wherein the nucleic acid vector is a naked nucleic acid vector.

53. The method of any one of embodiments 47-52, wherein the nucleic acid vector is encapsulated in, or associated with, a particulate structure.

54. The method of embodiment 53, wherein the particulate structure is a liposome.

55. The method of any one of embodiments 47-54, wherein the nucleic acid vector is a DNA vector.

56. The method of embodiment 55, wherein the DNA vector is a synthetic DNA vector.

57. The method of embodiment 56, wherein the synthetic DNA vector is a synthetic circular DNA vector.

58. The method of any one of embodiments 47-57, wherein the therapeutic sequence has a 5′ end and a 3′ end, wherein the 5′ end is connected to the 3′ end by a non-bacterial sequence corresponding to a restriction enzyme cut site overhang.

59. The method of any one of embodiments 47-58, wherein the expression level of the therapeutic sequence from the synthetic circular DNA vector is greater than expression level of a therapeutic sequence from a reference plasmid DNA vector under analogous conditions.

60. The method of embodiment 59, wherein the expression level of the therapeutic sequence from the synthetic circular DNA vector is at least 10% greater than expression level of a therapeutic sequence from a reference plasmid DNA vector under analogous conditions.

61. The method of any one of embodiments 47-60, wherein the expression persistence of the therapeutic sequence from the synthetic circular DNA vector is greater than expression persistence of a therapeutic sequence from a reference plasmid DNA vector under analogous conditions.

62. The method of any one of embodiments 47-61, wherein the therapeutic sequence comprises a therapeutic protein-encoding sequence.

63. The method of embodiment 62, wherein the therapeutic protein is CFTR.

64. The method of any one of embodiments 47-63, wherein the therapeutic sequence comprises one or more regulatory elements.

65. The method of any one of embodiments 47-61, wherein the therapeutic sequence comprises a therapeutic nucleic acid.

66. The method of any one of embodiments 47-65, wherein the target airway cell is an airway epithelial cell.

67. The method of any one of embodiments 47-66, wherein the target airway cell is a lung cell.

68. The method of any one of embodiments 47-67, wherein the target airway cell is a nasal epithelial cell, a tracheal cell, bronchial cell, bronchiolar cell, or an alveolar cell.

69. The method of any one of embodiments 47-68, wherein the conditions suitable for electrotransfer of the therapeutic agent into the target airway cell comprise a voltage at the target lung cell of less than 2,400 V.

70. The method of embodiment 69, wherein the voltage at the target airway cell is between 100 V and 2,400 V.

71. The method of any one of embodiments 47-70, wherein 1-6 pulses of electrical energy are transmitted.

72. The method of any one of embodiments 47-71, wherein the total number of pulses of electrical energy are transmitted within 1-20 seconds.

73. The method of any one of embodiments 47-72, wherein a single pulse of electrical energy is transmitted.

74. The method of any one of embodiments 47-73, wherein the one or more pulses of electrical energy are square waveforms.

75. The method of any one of embodiments 47-74, wherein the one or more pulses of electrical energy have an amplitude from 100 V to 5,000 V.

76. The method of any one of embodiments 47-75, wherein each of the pulses of electrical energy is from 0.01 to 200 milliseconds in duration.

77. The method of embodiment 76, wherein each of the pulses of electrical energy is from 10 to 50 milliseconds in duration.

78. The method of embodiment 77, wherein the total duration of all of the pulses of electrical energy is from 10 to 50 milliseconds.

79. The method of any one of embodiments 49-78, wherein the electrode is a monopolar electrode.

80. The method of any one of embodiments 49-78, wherein the electrode is a bipolar electrode.

81. The method of any one of embodiments 47-80, wherein the electrode is within 10 cm of the target airway cell.

82. The method of embodiment 81, wherein the electrode is within 1 cm of the target airway cell.

83. The method of any one of embodiments 47-82, wherein the target airway cell has a mutation in a gene correctable by the therapeutic sequence.

84. The method of any one of embodiments 47-83, wherein the airway lumen is trachea, bronchi, or bronchiole.

85. The method of any one of embodiments 47-84, wherein the individual has been diagnosed with a disease or disorder.

86. The method of embodiment 85, wherein the disease or disorder is a monogenic respiratory disease.

87. The method of embodiment 86, wherein the monogenic respiratory disease is cystic fibrosis.

88. The method of any one of embodiments 47-87, wherein the synthetic circular DNA vector has been administered to the individual locally.

89. The method of embodiment 88, wherein the local administration was intranasal administration or intramuscular administration.

90. The method of embodiment 88, wherein the local administration was by flood, spray, or aerosolization of the synthetic circular DNA vector.

91. The method of any one of embodiments 47-90, wherein the synthetic circular DNA vector has been administered to the individual systemically.

92. The method of embodiment 91, wherein the systemic administration was intravenous administration.

93. The method of any one of embodiments 47-92, wherein the method further comprises administering the synthetic circular DNA vector.

94. The method of any one of embodiments 49-93, wherein the partial airway epithelial ablation therapy was performed using a pulsed electric field.

95. The method of any one of embodiments 47-94, wherein the individual is a human.

96. A method of treating a disease or disorder in an individual, the method comprising:
(i) positioning an electrode in electrical communication with target airway cells of the individual, wherein the target airway cells are exposed to a therapeutically effective amount of a synthetic circular DNA vector comprising a therapeutic sequence; and
(ii) transmitting one or more pulses of electrical energy through the electrode at conditions suitable for electrotransfer of the synthetic circular DNA vector into the target airway cells, thereby expressing the therapeutic sequence and treating the disease or disorder.

97. The method of embodiment 96, wherein the synthetic circular DNA vector comprises a therapeutic sequence having a 5′ end and a 3′ end, wherein the 5′ end is connected to the 3′ end by a non-bacterial sequence.

98. The method of embodiment 97, wherein the non-bacterial sequence corresponds to a restriction enzyme cut site overhang.

99. The method of any one of embodiments 96-98, wherein the synthetic circular DNA vector is a naked synthetic circular DNA vector.

100. The method of any one of embodiments 96-99, wherein the synthetic circular DNA vector is encapsulated in, or associated with, a particulate structure.

101. The method of embodiment 100, wherein the particulate structure is a liposome.

102. The method of any one of embodiments 96-101, wherein the therapeutic sequence comprises a therapeutic protein-encoding sequence.

103. The method of embodiment 102, wherein the therapeutic protein is CFTR.

104. The method of any one of embodiments 96-103, wherein the therapeutic sequence comprises one or more regulatory elements.

105. The method of any one of embodiments 96-104, wherein the therapeutic sequence comprises a therapeutic nucleic acid.

106. The method of any one of embodiments 96-105, wherein the target airway cell is an airway epithelial cell.

107. The method of any one of embodiments 96-106, wherein the target airway cell is a lung cell.

108. The method of any one of embodiments 96-107, wherein the target airway cell is a nasal epithelial cell, a tracheal cell, bronchial cell, bronchiolar cell, or an alveolar cell.

109. The method of any one of embodiments 96-108, wherein the conditions suitable for electrotransfer of the therapeutic agent into the target airway cell comprise a voltage at the target lung cell of less than 2,400 V.

110. The method of embodiment 109, wherein the voltage at the target airway cell is between 100 V and 2,400 V.

111. The method of any one of embodiments 96-110, wherein 1-6 pulses of electrical energy are transmitted.

112. The method of any one of embodiments 96-111, wherein the total number of pulses of electrical energy are transmitted within 1-20 seconds.

113. The method of any one of embodiments 96-112, wherein a single pulse of electrical energy is transmitted.

114. The method of any one of embodiments 96-113, wherein the one or more pulses of electrical energy are square waveforms.

115. The method of any one of embodiments 96-114, wherein the one or more pulses of electrical energy have an amplitude from 100 V to 5,000 V.

116. The method of any one of embodiments 96-115, wherein each of the pulses of electrical energy is from 0.01 to 200 milliseconds in duration.

117. The method of any one of embodiments 96-116, wherein each of the pulses of electrical energy is from 10 to 50 milliseconds in duration.

118. The method of any one of embodiments 96-117, wherein the total duration of all of the pulses of electrical energy is from 10 to 50 milliseconds.

119. The method of any one of embodiments 96-118, wherein the electrode is a monopolar electrode.

120. The method of any one of embodiments 96-118, wherein the electrode is part of a bipolar electrode configuration.

121. The method of any one of embodiments 96-120, wherein the electrode is within 10 cm of the target airway cell.

122. The method of embodiment 121, wherein the electrode is within 1 cm of the target airway cell.

123. The method of any one of embodiments 96-122, wherein the expression level of the therapeutic sequence from the synthetic circular DNA vector is greater than expression level of a therapeutic sequence from a reference plasmid DNA vector under analogous conditions.

124. The method of embodiment 123, wherein the expression level of the therapeutic sequence from the synthetic circular DNA vector is at least 10% greater than expression level of a therapeutic sequence from a reference plasmid DNA vector under analogous conditions.

125. The method of any one of embodiments 96-124, wherein the expression persistence of the therapeutic sequence from the synthetic circular DNA vector is greater than expression persistence of a therapeutic sequence from a reference plasmid DNA vector under analogous conditions.

126. The method of any one of embodiments 96-125, wherein the target airway cell has a mutation in a gene correctable by the therapeutic sequence.

127. The method of any one of embodiments 96-126, wherein step (i) comprises inserting a catheter comprising the electrode into an airway lumen of the individual.

128. The method of embodiment 127, wherein the airway lumen is trachea, bronchi, or bronchiole.

129. The method of any one of embodiments 96-128, wherein the disease or disorder is a monogenic respiratory disease.

130. The method of embodiment 129, wherein the monogenic respiratory disease is cystic fibrosis.

131. The method of any one of embodiments 96-130, wherein the synthetic circular DNA vector has been administered to the individual locally.

132. The method of embodiment 131, wherein the local administration was intranasal administration or intramuscular administration.

133. The method of embodiment 131, wherein the local administration was by flood, spray, or aerosolization of the synthetic circular DNA vector.

134. The method of any one of embodiments 96-131, wherein the synthetic circular DNA vector has been administered to the individual systemically.

135. The method of embodiment 134, wherein the systemic administration was intravenous administration.

136. The method of any one of embodiments 96-135, wherein the method further comprises administering the synthetic circular DNA vector.

137. The method of any one of embodiments 96-136, wherein partial airway epithelial ablation therapy was administered prior to administration of the synthetic circular DNA vector.

138. The method of embodiment 137, wherein the partial airway epithelial ablation therapy was administered using a pulsed electric field.

139. The method of embodiment 137 or 138, wherein the method further comprises administering the partial airway epithelial ablation therapy.

140. A method of treating cystic fibrosis in an individual, the method comprising:
(i) inserting a catheter comprising an electrode into an airway lumen of the individual;
(ii) positioning the electrode in electrical communication with target airway cells of the individual, wherein the target airway cells are exposed to a therapeutically effective amount of a synthetic circular DNA vector encoding CFTR; and
(iii) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

141. A method of treating cystic fibrosis in an individual, the method comprising:
(i) administering to the individual a therapeutically effective amount of a synthetic circular DNA vector encoding CFTR;
(ii) inserting a catheter comprising an electrode into an airway lumen of the individual;
(iii) positioning the electrode in electrical communication with target airway cells of the individual, wherein the target airway cells are exposed to the synthetic circular DNA vector encoding CFTR; and
(iv) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

142. A method of treating cystic fibrosis in an individual, the method comprising:
(i) administering partial airway epithelial ablation therapy to the individual;
(ii) administering to the individual a therapeutically effective amount of a synthetic circular DNA vector encoding CFTR;
(iii) inserting a catheter comprising an electrode into an airway lumen of the individual;
(iv) positioning the electrode in electrical communication with target airway cells of the individual, wherein the target airway cells are exposed to the synthetic circular DNA vector encoding CFTR; and
(v) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

143. A synthetic circular DNA vector for use in a method of expressing a therapeutic sequence in a target airway cell, the method comprising:
(i) exposing the target airway cell to the synthetic circular DNA vector, wherein the synthetic circular DNA vector comprises the therapeutic sequence, wherein the target airway cell is in electrical communication with an electrode; and
(ii) transmitting one or more pulses of electrical energy through the electrode at conditions suitable for electrotransfer of the synthetic circular DNA vector into the target airway cell, thereby expressing the therapeutic sequence.

144. The synthetic circular DNA vector for use of embodiment 143, wherein the synthetic circular DNA vector is a naked synthetic circular DNA vector.

145. The synthetic circular DNA vector for use of embodiment 143, wherein the synthetic circular DNA vector is encapsulated in, or associated with, a particulate structure.

146. The synthetic circular DNA vector for use of embodiment 145, wherein the particulate structure is a liposome.

147. The synthetic circular DNA vector for use of any one of embodiments 1-146, wherein the therapeutic sequence comprises a therapeutic protein-encoding sequence.

148. The synthetic circular DNA vector for use of embodiment 147, wherein the therapeutic protein is cystic fibrosis transmembrane receptor (CFTR).

149. The synthetic circular DNA vector for use of any one of embodiments 143-148, wherein the therapeutic sequence comprises one or more regulatory elements.

150. The synthetic circular DNA vector for use of any one of embodiments 143-149, wherein the therapeutic sequence comprises a therapeutic nucleic acid.

151. The synthetic circular DNA vector for use of any one of embodiments 143-150, wherein the target airway cell is an airway epithelial cell.

152. The synthetic circular DNA vector for use of any one of embodiments 143-151, wherein the target airway cell is a lung cell.

153. The synthetic circular DNA vector for use of any one of embodiments 143-152, wherein the target airway cell is a nasal epithelial cell, a tracheal cell, bronchial cell, bronchiolar cell, or an alveolar cell.

154. The synthetic circular DNA vector for use of any one of embodiments 143-153, wherein the conditions suitable for electrotransfer of the therapeutic agent into the target airway cell comprise a voltage at the target lung cell of less than 2,400 V.

155. The synthetic circular DNA vector for use of embodiment 154, wherein the voltage at the target airway cell is between 100 V and 2,400 V.

156. The synthetic circular DNA vector for use of embodiment 155, wherein the voltage at the target airway cell is between 1,600 V and 2,400 V, or is about 1,600 V.

157. The synthetic circular DNA vector for use of any one of embodiments 143-156, wherein 1-6 pulses of electrical energy are transmitted, or wherein 2 pulses of electrical energy are transmitted, or wherein a single pulse of electrical energy is transmitted.

158. The synthetic circular DNA vector for use of any one of embodiments 143-157, wherein the total number of pulses of electrical energy are transmitted within 1-20 seconds.

159. The synthetic circular DNA vector for use of any one of embodiments 143-158, wherein each of the pulses of electrical energy is from 0.01 to 200 milliseconds in duration.

160. The synthetic circular DNA vector for use of embodiment 159, wherein each of the pulses of electrical energy is from 10 to 50 milliseconds in duration.

161. The synthetic circular DNA vector for use of any one of embodiments 143-160, wherein the electrode is a monopolar electrode.

162. The synthetic circular DNA vector for use of any one of embodiments 143-160, wherein the electrode is part of a bipolar electrode configuration.

163. The synthetic circular DNA vector for use of any one of embodiments 143-162, wherein the electrode is within 1 cm of the target airway cell.

164. The synthetic circular DNA vector for use of any one of embodiments 143-163, wherein the expression level of the therapeutic sequence from the synthetic circular DNA vector is at least 10% greater than expression level of a therapeutic sequence from a reference plasmid DNA vector under analogous conditions.

165. The synthetic circular DNA vector for use of any one of embodiments 143-164, wherein the expression persistence of the therapeutic sequence from the synthetic circular DNA vector is greater than expression persistence of a therapeutic sequence from a reference plasmid DNA vector under analogous conditions.

166. The synthetic circular DNA vector for use of any one of embodiments 143-165, wherein the target airway cell has a mutation in a gene correctable by the therapeutic sequence.

167. The synthetic circular DNA vector for use of any one of embodiments 143-166, wherein step (ii) is performed in situ in an individual by inserting a catheter comprising the electrode into an airway lumen of the individual.

168. The synthetic circular DNA vector for use of embodiment 167, wherein the airway lumen is trachea, bronchi, or bronchiole.

169. The synthetic circular DNA vector for use of embodiment 167 or 168, wherein the individual has been diagnosed with a disease or disorder.

170. The synthetic circular DNA vector for use of embodiment 169, wherein the disease or disorder is a monogenic respiratory disease.

171. The synthetic circular DNA vector for use of embodiment 170, wherein the monogenic respiratory disease is cystic fibrosis.

172. The synthetic circular DNA vector for use of any one of embodiments 143-171, wherein the synthetic circular DNA vector has been administered to the individual locally.

173. The synthetic circular DNA vector for use of embodiment 172, wherein the local administration was intranasal administration or intramuscular administration.

174. The synthetic circular DNA vector for use of embodiment 172, wherein the local administration was by flood, spray, or aerosolization of the synthetic circular DNA vector.

175. The synthetic circular DNA vector for use of any one of embodiments 143-171, wherein the synthetic circular DNA vector has been administered to the individual systemically.

176. The synthetic circular DNA vector for use of embodiment 175, wherein the systemic administration was intravenous administration.

177. The synthetic circular DNA vector for use of any one of embodiments 143-176, wherein the method further comprises administering the synthetic circular DNA vector.

178. The synthetic circular DNA vector for use of any one of embodiments 1-177, wherein partial airway epithelial ablation therapy has preceded the administration of the synthetic circular DNA vector.

179. The synthetic circular DNA vector for use of embodiment 178, wherein the partial airway epithelial ablation was administered using a pulsed electric field.

180. The synthetic circular DNA vector for use of any one of embodiments 143-179, wherein the method further comprises administering the partial airway epithelial ablation therapy.

181. The method of any one of embodiments 143-180, wherein the target airway cell is a human cell.

182. A therapeutic agent for use in a method of delivering the therapeutic agent to a target airway cell in an individual that has been treated with a partial airway epithelial ablation therapy, the method comprising:
(i) inserting a catheter comprising an electrode into an airway lumen of the individual;
(ii) positioning the electrode within a target region of the airway lumen comprising the target airway cell and the therapeutic agent; and
(iii) while the electrode is within the target region, transmitting one or more pulses of electrical energy through the electrode at conditions suitable for electrotransfer of the therapeutic agent into the target airway cell.

183. The therapeutic agent for use of embodiment 182, wherein the therapeutic agent comprises a nucleic acid vector comprising a therapeutic sequence, optionally, wherein the nucleic acid vector is a non-viral nucleic acid vector, is a naked nucleic acid vector, or is encapsulated in, or associated with, a particulate structure.

184. A synthetic circular DNA vector for use in a method of treating a disease or disorder in an individual, the method comprising:
(i) positioning an electrode in electrical communication with target airway cells of the individual, wherein the target airway cells are exposed to a therapeutically effective amount of the synthetic circular DNA vector, wherein the synthetic circular DNA vector comprises a therapeutic sequence; and
(ii) transmitting one or more pulses of electrical energy through the electrode at conditions suitable for electrotransfer of the synthetic circular DNA vector into the target airway cells, thereby expressing the therapeutic sequence and treating the disease or disorder.

185. A synthetic circular DNA vector for use in a method of treating cystic fibrosis in an individual, the method comprising:
(i) inserting a catheter comprising an electrode into an airway lumen of the individual;
(ii) positioning the electrode in electrical communication with target airway cells of the individual, wherein the target airway cells are exposed to a therapeutically effective amount of the synthetic circular DNA vector, wherein the synthetic circular DNA vector encodes CFTR; and
(iii) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

186. A synthetic circular DNA vector for use in a method of treating cystic fibrosis in an individual, the method comprising:
(i) administering to the individual a therapeutically effective amount of the synthetic circular DNA vector, wherein the synthetic circular DNA vector encodes CFTR;
(ii) inserting a catheter comprising an electrode into an airway lumen of the individual;
(iii) positioning the electrode in electrical communication with target airway cells of the individual, wherein the target airway cells are exposed to the synthetic circular DNA vector encoding CFTR; and
(iv) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

187. A synthetic circular DNA vector for use in a method of treating cystic fibrosis in an individual, the method comprising:
(i) administering partial airway epithelial ablation therapy to the individual;
(ii) administering to the individual a therapeutically effective amount of the synthetic circular DNA vector, wherein the synthetic circular DNA vector encodes CFTR;
(iii) inserting a catheter comprising an electrode into an airway lumen of the individual;
(iv) positioning the electrode in electrical communication with target airway cells of the individual, wherein the target airway cells are exposed to the synthetic circular DNA vector encoding CFTR; and
(v) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

What is claimed is:

1. A method of expressing a therapeutic sequence in a target airway cell, the method comprising:
(i) exposing the target airway cell to a synthetic circular DNA vector comprising the therapeutic sequence, wherein the target airway cell is in electrical communication with an electrode; and
(ii) transmitting one or more pulses of electrical energy through the electrode at conditions suitable for electrotransfer of the synthetic circular DNA vector into the target airway cell, thereby expressing the therapeutic sequence.

2. The method of claim 1, wherein the synthetic circular DNA vector comprises a therapeutic sequence having a 5′ end and a 3′ end, wherein the 5′ end is connected to the 3′ end by a non-bacterial sequence.

3. The method of claim 2, wherein the non-bacterial sequence corresponds to a restriction enzyme cut site overhang.

4. The method of claim 1, wherein the synthetic circular DNA vector is a naked synthetic circular DNA vector.

5. The method of claim 1, wherein the synthetic circular DNA vector is encapsulated in, or associated with, a particulate structure.

6. The method of claim 5, wherein the particulate structure is a liposome.

7. The method of claim 1, wherein the therapeutic sequence comprises a therapeutic protein-encoding sequence.

8. The method of claim 7, wherein the therapeutic protein is cystic fibrosis transmembrane receptor (CFTR).

9. The method of claim 1, wherein the target airway cell is an airway epithelial cell.

10. The method of claim 1, wherein the conditions suitable for electrotransfer of the therapeutic agent into the target airway cell comprise a voltage at the target lung cell of less than 2,400 V.

11. The method of any one of claims 1-10, wherein 1-6 pulses of electrical energy are transmitted.

12. The method of any one of claims 1-11, wherein the total number of pulses of electrical energy are transmitted within 1-20 seconds.

13. The method of claim 11, wherein a single pulse of electrical energy is transmitted.

14. The method of any one of claims 1-13, wherein the one or more pulses of electrical energy have an amplitude from 100 V to 5,000 V.

15. The method of any one of claims 1-14, wherein each of the pulses of electrical energy is from 0.01 to 200 milliseconds in duration.

16. The method of claim 15, wherein each of the pulses of electrical energy is from 10 to 50 milliseconds in duration.

17. The method of any one of claims 1-16, wherein the expression level of the therapeutic sequence from the synthetic circular DNA vector is greater than expression level of a therapeutic sequence from a reference plasmid DNA vector under analogous conditions.

18. The method of any one of claims 1-17, wherein the target airway cell has a mutation in a gene correctable by the therapeutic sequence.

19. The method of any one of claims 1-18, wherein step (ii) is performed in situ in an individual by inserting a catheter comprising the electrode into an airway lumen of the individual.

20. The method of claim 19, wherein the airway lumen is trachea, bronchi, or bronchiole.

21. The method of claim 19 or 20, wherein the individual has been diagnosed with a disease or disorder.

22. The method of claim 21, wherein the disease or disorder is a monogenic respiratory disease.

23. The method of claim 22, wherein the monogenic respiratory disease is cystic fibrosis.

24. The method of any one of claims 1-23, wherein the synthetic circular DNA vector has been administered to the individual locally.

25. The method of claim 24, wherein the local administration was intranasal administration or intramuscular administration.

26. The method of claim 24, wherein the local administration was by flood, spray, or aerosolization of the synthetic circular DNA vector.

27. The method of any one of claims 1-23, wherein the synthetic circular DNA vector has been administered to the individual systemically.

28. The method of any one of claims 1-27, wherein the method further comprises administering the synthetic circular DNA vector.

29. The method of any one of claims 1-28, wherein partial airway epithelial ablation therapy has preceded the administration of the synthetic circular DNA vector.

30. The method of claim 29, wherein the partial airway epithelial ablation was administered using a pulsed electric field.

31. The method of any one of claims 1-30, wherein the method further comprises administering the partial airway epithelial ablation therapy.

32. The method of any one of claims 1-31, wherein the target airway cell is a human cell.

33. A method of delivering a therapeutic agent to a target airway cell in an individual, the method comprising:
(i) inserting a catheter comprising a monopolar electrode into an airway lumen of the individual;
(ii) positioning the electrode within a target region of the airway lumen comprising the target airway cell and the therapeutic agent;
(iii) while the electrode is within the target region, transmitting one or more pulses of electrical energy through the electrode at conditions suitable for electrotransfer of the therapeutic agent into the target airway cell.

34. The method of claim 33, wherein the individual has been treated with a partial airway epithelial ablation therapy.

35. A method of delivering a therapeutic agent to a target airway cell in an individual that has been treated with a partial airway epithelial ablation therapy, the method comprising:
(i) inserting a catheter comprising an electrode into an airway lumen of the individual;
(ii) positioning the electrode within a target region of the airway lumen comprising the target airway cell and the therapeutic agent; and
(iii) while the electrode is within the target region, transmitting one or more pulses of electrical energy through the electrode at conditions suitable for electrotransfer of the therapeutic agent into the target airway cell.

36. A method of treating a disease or disorder in an individual, the method comprising:
(i) positioning an electrode in electrical communication with target airway cells of the individual, wherein the target airway cells are exposed to a therapeutically effective amount of a synthetic circular DNA vector comprising a therapeutic sequence; and
(ii) transmitting one or more pulses of electrical energy through the electrode at conditions suitable for electrotransfer of the synthetic circular DNA vector into the target airway cells, thereby expressing the therapeutic sequence and treating the disease or disorder.

37. The method of claim 36, wherein the expression level of the therapeutic sequence from the synthetic circular DNA vector is greater than expression level of a therapeutic sequence from a reference plasmid DNA vector under analogous conditions.

38. The method of claim 36 or 37, wherein the expression persistence of the therapeutic sequence from the synthetic circular DNA vector is greater than expression persistence of a therapeutic sequence from a reference plasmid DNA vector under analogous conditions.

39. The method of any one of claims 36-38, wherein the target airway cell has a mutation in a gene correctable by the therapeutic sequence.

40. The method of any one of claims 36-39, wherein the disease or disorder is a monogenic respiratory disease.

41. The method of claim 40, wherein the monogenic respiratory disease is cystic fibrosis.

42. The method of any one of claims 36-41, wherein partial airway epithelial ablation therapy was administered prior to administration of the synthetic circular DNA vector.

43. The method of claim 42, wherein the partial airway epithelial ablation therapy was administered using a pulsed electric field.

44. A method of treating cystic fibrosis in an individual, the method comprising:
(i) inserting a catheter comprising an electrode into an airway lumen of the individual;
(ii) positioning the electrode in electrical communication with target airway cells of the individual, wherein the target airway cells are exposed to a therapeutically effective amount of a synthetic circular DNA vector encoding CFTR; and
(iii) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

45. A method of treating cystic fibrosis in an individual, the method comprising:
(i) administering to the individual a therapeutically effective amount of a synthetic circular DNA vector encoding CFTR;
(ii) inserting a catheter comprising an electrode into an airway lumen of the individual;
(iii) positioning the electrode in electrical communication with target airway cells of the individual, wherein the target airway cells are exposed to the synthetic circular DNA vector encoding CFTR; and
(iv) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.

46. A method of treating cystic fibrosis in an individual, the method comprising:
(i) administering partial airway epithelial ablation therapy to the individual;
(ii) administering to the individual a therapeutically effective amount of a synthetic circular DNA vector encoding CFTR;
(iii) inserting a catheter comprising an electrode into an airway lumen of the individual;
(iv) positioning the electrode in electrical communication with target airway cells of the individual, wherein the target airway cells are exposed to the synthetic circular DNA vector encoding CFTR; and
(v) transmitting one to six pulses of electrical energy through the electrode at a voltage at the target lung cell between 100 V and 2,400 V, wherein the total duration of the pulses of electrical energy is from 10 to 50 milliseconds, thereby expressing the CFTR and treating the cystic fibrosis.
